(19) United States
(12) Patent Application Publication
(10) Pub. No.: US 2004/0073229 A1
(43) Pub. Date: Apr. 15, 2004

(54) Thoracic spine posterior transpedicular screw finder

(76) Inventor: Lin-Min Yang, Taichung (TW)

Correspondence Address: Liberty Patent and Trademark Office, P.O. Box 590, Taichung City, Taichung, 400, TW

(21) Appl. No.: 10/268,893

(22) Filed: Oct. 11, 2002

(51) Int. Cl. A61B 17/58

(52) U.S. Cl. 606/104

(57) ABSTRACT

The present invention relates to a thoracic spine posterior transpedicular screw finder that guides a drilling device quickly to the exact position for drilling purpose onto vertebra, secures the drilling process and prevents it from deviation in spinal instrumentation with the transpedicular fixation of spinal operation.

### BACKGROUND OF THE INVENTION

At present, posterior instrumentation with transpedicular screws or hooks system is common in spinal surgery. To perform a posterior spinal instrumentation, the fixation process is made by inserting a vertebral screw which is called ‘transpedicular screw’ through the pedicle into the body, or a hook into the lamina. The former is usually used in lumbar spinal surgery, while the latter in thoracic spinal surgery. The fixation and strength is best while inserting a transpedicular screw through the pedicle into the body, but at the present, it is uncommon used in the thoracic spinal surgery for higher difficult technology. The new innovation provides specially designed tools for the operator to insert the thoracic spine transpedicular screw correctly, safely and easily, instead of the less effective hooks.

The transpedicular screws are mainly used in the lumbar vertebral surgery, while the inlet for the vertebral screw is easily to identify by referent anatomy and the space of the pedicle is sufficient for the vertebral screw. While in the thoracic spinal surgery, the referent anatomy is not clear enough to find correct position exactly for transpedicular screw and the space of the pedicle for the vertebral screw is guarded, most surgeons choose the safer method with lamina or pedicle hooks onto the thoracic vertebra 19. But the result is less satisfactory due to less fixation strength and more complications, such as dislodgement of the hooks. Because of the poorer fixation of a hook, the result is less satisfactory. Therefore, in order for better result, there still are some highly skilled physicians who are still willing to use transpedicular screws in the thoracic spinal surgery.

The conventional method for identification for a thoracic pedicle screw inlet was mainly determined by the anatomical landmarkings. The anatomical landmarkings for identification of the thoracic spine transpedicular screw inlet is not clear enough, even a skillful surgeon could not determine the pedicle screw inlet precisely. Even a little error could result in catastrophic complications. This is why transpedicular screw is not widely used in thoracic spine surgery. By our new innovations, transpedicular screws could be inserted easily and safely in the thoracic spinal surgery, and provide better stability as compared with conventional thoracic spinal hooks system.

### SUMMARY OF THE INVENTION

Therefore, the inventor has devoted in research to seek improvement on the shortcomings of the conventional method, for application of thoracic transpedicular screw and after extended research, has come up with the present invention. The primary objective of the invention is to provide a posterior transpedicular screw finder that is capable of speedily and exactly guide a device to find the inlet for vertebral screw correctly and avoid any deviation, comprising a locating unit, a rod body and a grip part. Of which, the locating unit has a recess with its opening facing forward at the bottom thereof, forming a locating block that extends forward at a bottom thereof, the front of the locating block having a locating fork with an arched inside. The front of the locating unit has a duct at its front, the center of the duct being located on a line extending from a center of the locating fork. The rod body extends outwardly from one side of the locating unit and bent upwards to become an “L” shape, at its end being a grip part that can be gripped by a user, thereby the locating fork can be mounted onto the pedicle of the vertebra to prevent the duct of the locating unit from sliding left and right. The recess mounted onto the bottom of the lamina of the thoracic vertebra keeps the duct of the locating unit from moving up and down. By mounting the arch on the inside of the locating fork onto the pedicle of the thoracic vertebra, it will automatically guide and locate the center position of the pedicle, so that it is automatically positioned on the center line of the locating groove, and relatively the center point of the duct positioned on top of the lamina falls onto the center position of the pedicle, thereby when the drilling device is inserted into the drill hole of the duct, it will drill a positioning hole squarely on top of the pedicle to facilitate inserting of the steel screw, thereby achieve the efficiency of speedy and precise guidance, locating and avoid deviation during drilling process.

For better understanding of the characteristics and technical contents of the present invention, please refer to the following description and drawings.

### BRIEF DESCRIPTION OF DRAWINGS

FIG. 1 is a perspective view of a conventional posterior thoracic vertebral instrumentation.

FIG. 2 is a section view for the approach of a conventional posterior thoracic vertebral instrumentation.

FIG. 3 is a section view of a conventional posterior thoracic vertebral instrumentation at the drilling with a steel screw.

FIG. 4 is a perspective view of the present invention.

FIG. 5 is a view for the approach of the invention when positioned on a thoracic vertebra.

FIG. 6 is a view for the approach of the invention positioned on a thoracic vertebra with the drilling device for insertion.

FIG. 7 is a section view of the invention positioned on a thoracic vertebra with the drilling device for insertion.

FIG. 8 is a view of the invention in application when viewed from another angle.

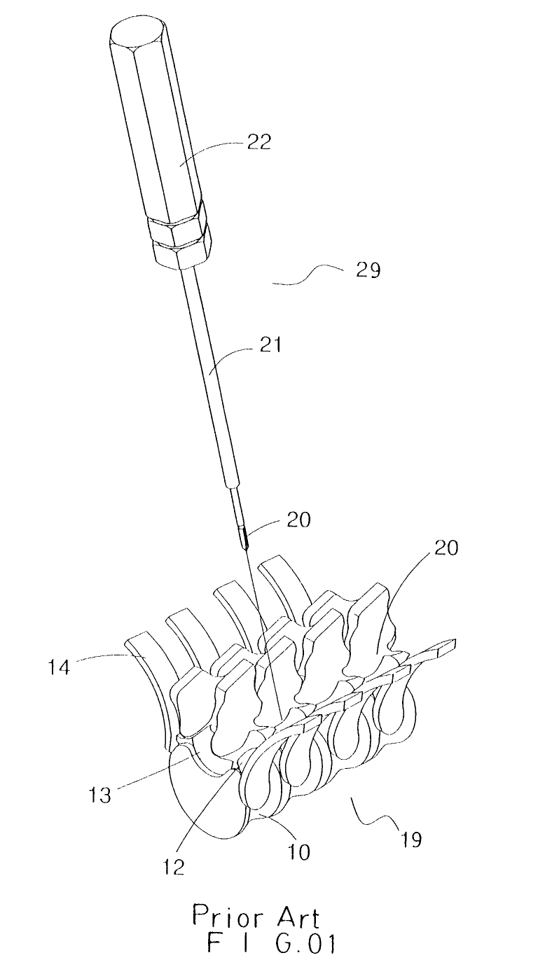

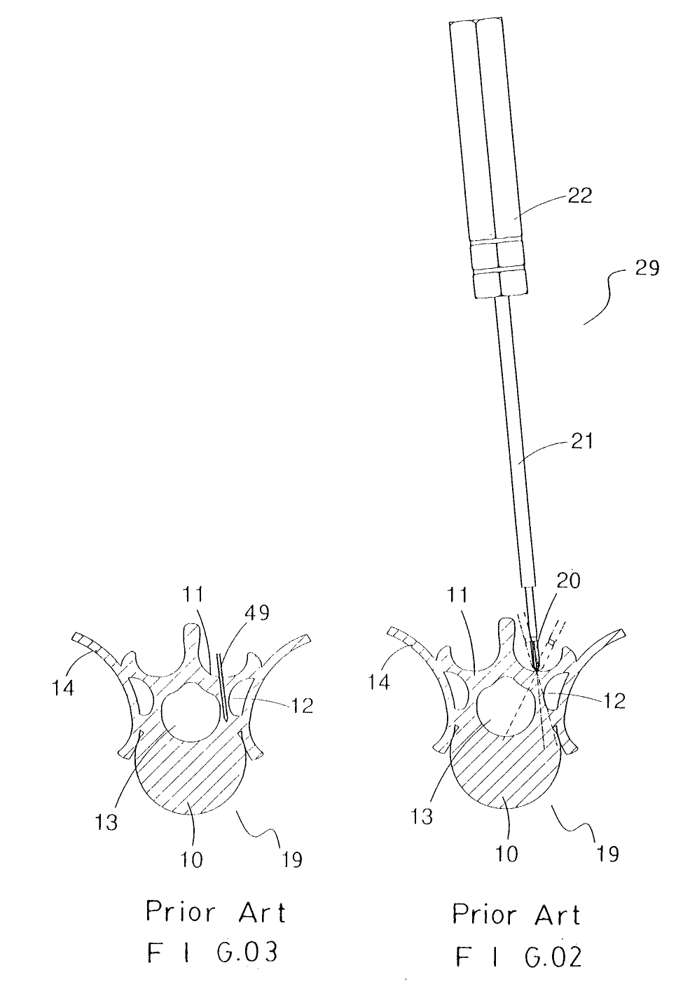

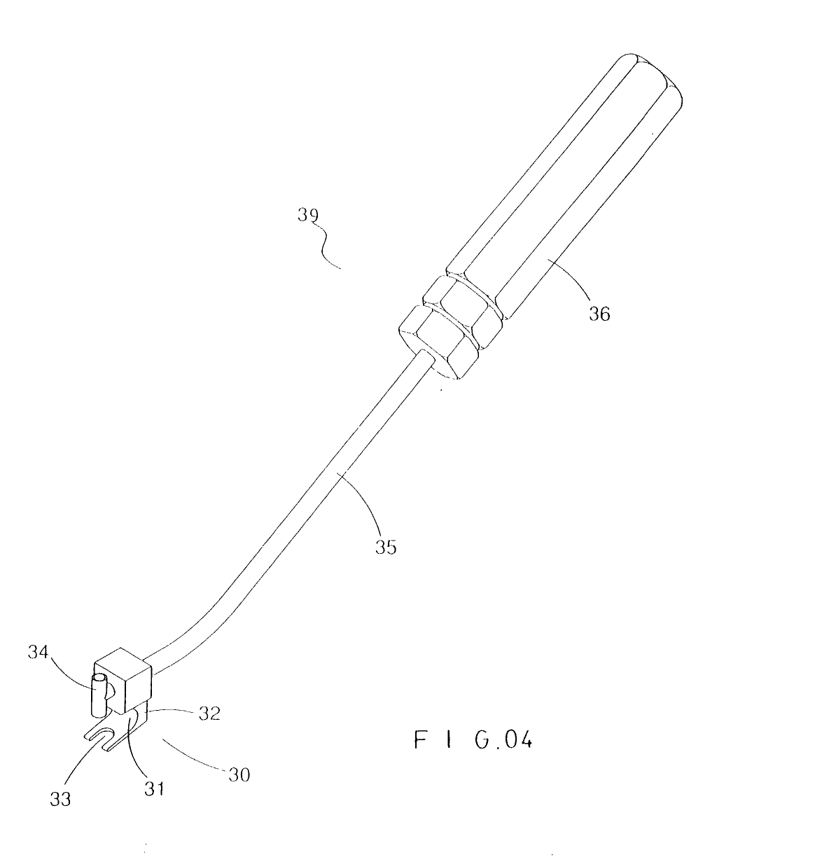

### BRIEF DESCRIPTION OF NUMERALS

| | | | |
|---|---|---|---|
| 19 | thoracic vertebra (e) | 10 | thoracic vertebral body |
| 11 | lamina | 12 | pedicle |
| 13 | vertebral cavity | 14 | rib |
| 29 | drilling device | 20 | drill bit |
| 21 | drill rod | 22 | handle |
| 39 | spinal column drill locator | 30 | locating unit |
| 31 | recess | 32 | locating block |
| 33 | locating fork | 34 | ducts |
| 35 | rod body | 36 | grip part |
| 49 | steel screw | | |

### DETAILED DESCRIPTION OF PREFERRED EMBODIMENTS

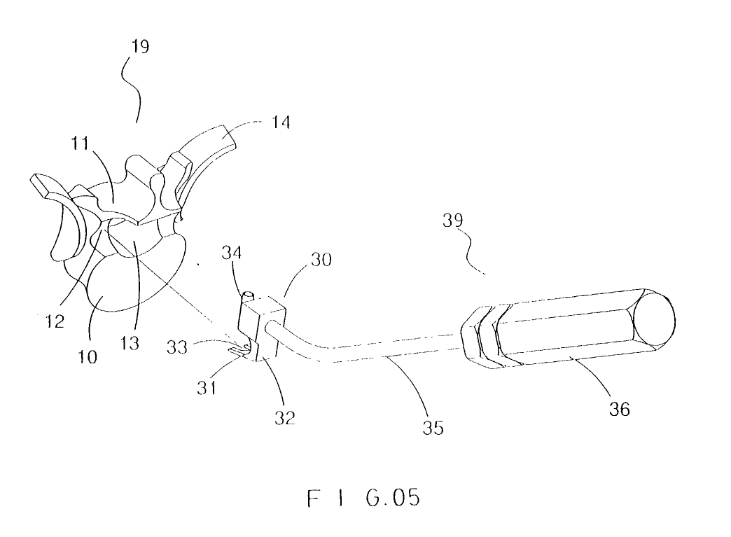

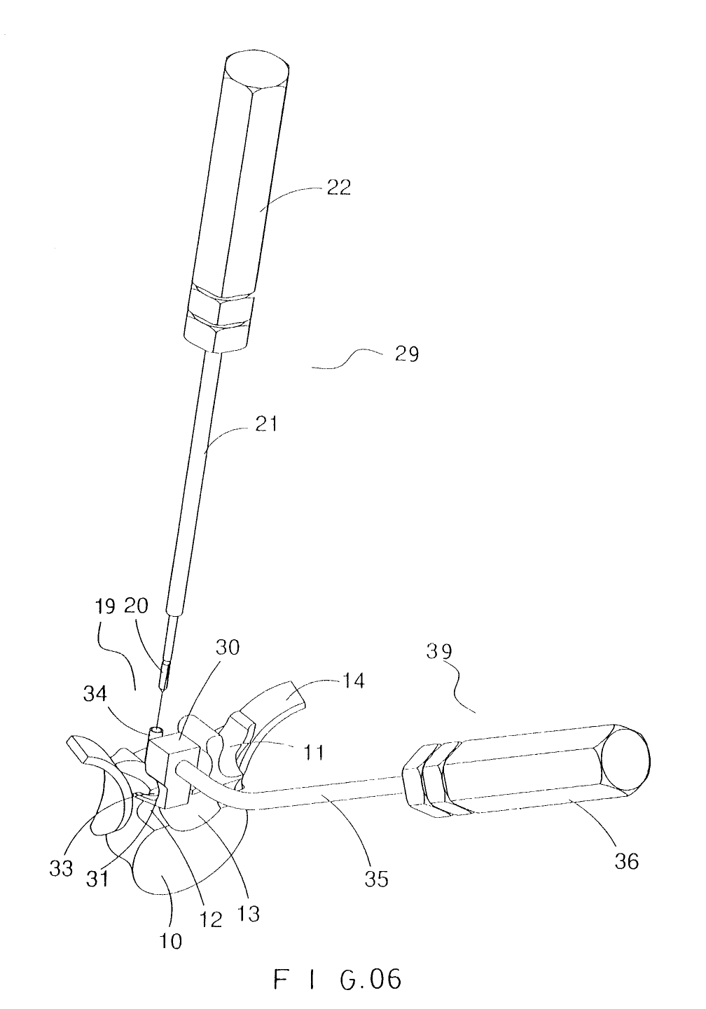

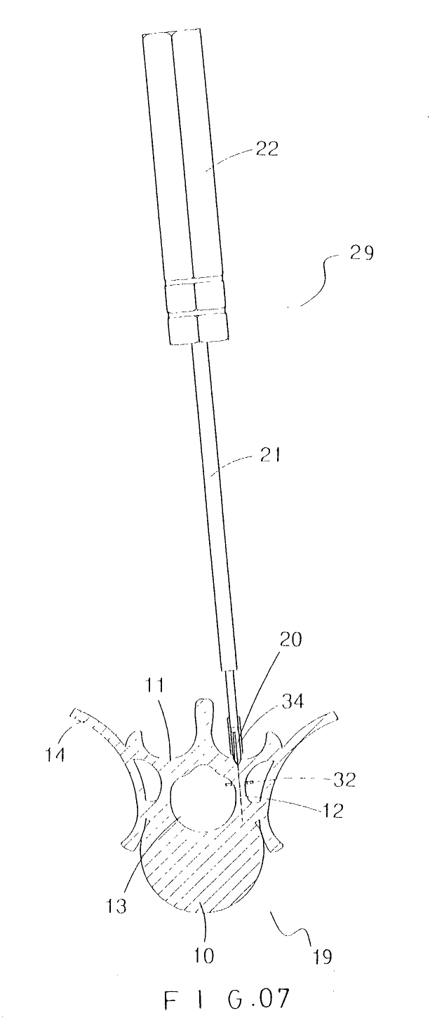

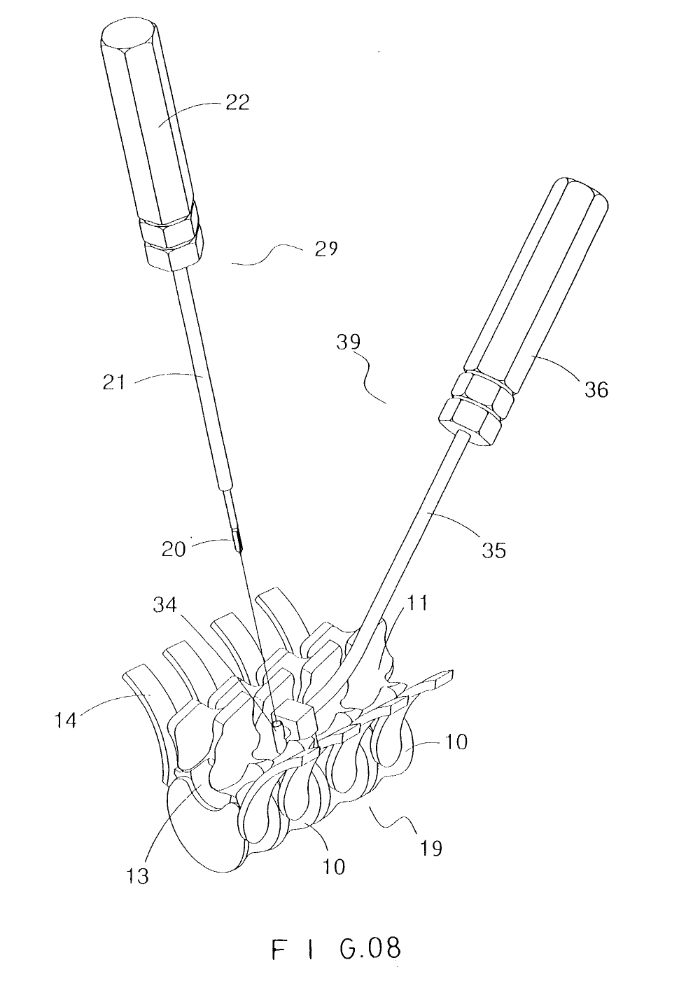

Please refer to FIG. 4 that illustrates a posterior transpedicular screw finder 39, comprising a locating unit 30, a rod body 35 and a grip part 36, wherein the locating unit 30 has a recess 31 opening to the front on a lower part thereof, so that its bottom part is shaped to protrude forward as a locating block 32. At the front part of the locating block 32 is a locating fork 33. At the front of the locating unit 30 is a duct 34 that accommodates the insertion of a drilling device 29. The center of the duck 34 is positioned on a line extending from the center of the locating fork 33. The rod body 35 extends outwardly from one side of the locating unit 30 and bends upwards to become an “L” shape, on its end is a grip part 36 that can be gripped by a user. Thereby, the locating fork 33 is mounted on a pedicle of a thoracic vertebra, and the duct can be speedily and accurately guide the drilling device into a drilling position, which is properly secured by the engagement of the locating fork 33 and the recess 31 onto the pedicle 12 and a lamina 11 of a vertebra. As shown in FIGS. 5 through 8, when the locating fork 33 of the locating unit 30 of the invention is mounted on the pedicle 12 on a thoracic vertebra body 10, the locating unit 30 is prevented from dislocating to its left or right side. Meanwhile, when the recess 31 is mounted to the bottom part of the lamina 11 of a thoracic vertebra 19, the locating unit 30 is also prevented from sliding up or down. Furthermore, when the arch of the locating fork 33 is pushed against the pedicle 12 of the thoracic vertebra 19, it will automatically lead to locate the center position of the pedicle 12, and automatically position the pedicle 12 on the centerline of the locating fork 33. In relation to that, the duct 34 at the front of the locating unit 30 is then positioned at the top of the center of the pedicle 12 of the lamina 11. Thereby, when the drilling device 29 is inserted inside the duct 34 to drill a hole, it will drill a position hole at a correct angle right on top of the pedicle 12, to facilitate the insertion of a steel screw 49 in the exact position.

Due to unique structural design of the invention, there are characteristics in application as follows:

1. Excellent locating: Since the invention relies on the arch on the inside of the locating fork 33 to automatically guide and locate the center position of the pedicle 12 and automatically position it on a center line of the locating fork 33 when the locating fork 33 is pressed against the pedicle 12 of the thoracic vertebra 19, and relatively, the duct 34 at the front of the locating unit 30 is positioned on a center extended line of the locating fork 33, therefore it is capable of promptly locating the optimum positioning spot. Furthermore, the locating fork 33 of the locating unit 30 is mounted onto the pedicle 12 of the thoracic vertebral body 10 to limit the locating unit 30 from misalignment. Meanwhile, the recess 31 is also mounted onto the bottom of the lamina 11 of the thoracic vertebra 19 to limit the locating unit 30 from up and down movement, therefore, the drilling device 29 can be inserted for drilling a hole on the thoracic vertebra 19, to achieve a better locating effect.

2. Simplified and speedy application and operation: Using the locating fork 33 of the locating unit 30 to promptly locate the center position of the pedicle 12, and using the locating fork 33 and recess 31 of the locating unit 30 to effectively engage in position, the drilling device 29 can be correctly aligned with the top of the center of the pedicle 12 of the thoracic vertebra 19, to drill a hole in the exact position, so that the steel screw 49 is squarely screwed into the pedicle 12 without harming other part. Therefore, the frequency of X-ray radiation and examination can be greatly reduced to minimize bad effects on human body. And, due to its significantly shortening of time involved in the process, all possible risks can be minimized in the operation.

To sum up, the sophisticated design of the invention has contributed to the aforementioned characteristics and applicability, and since similar products have never been seen or used in public, this application is filed.

What is claimed is:

1. A posterior transpedicular screw finder, comprising a locating unit, a rod body and a grip part, wherein the locating unit has a recess opening to the front at the lower part thereof, forming a locating block that extends to the front at the bottom part thereof, a front end of the locating block having an arched locating fork, at the front of the locating unit being a duct, the center of the duct being positioned on an line extending from the center of the locating fork, the rod body being extending outwardly from one side of the locating unit and bent upward in an “L” shape, on the end thereof being a grip part serving to be gripped by the user, thereby the user can mount the locating fork onto the pedicle of the thoracic vertebra, so the duct can speedily and precisely guide the drilling device to the position for drilling a hole, where the drilling process can be secured in a precise position because of the engagement of the locating fork and the recess onto the pedicle and the lamina of the thoracic vertebra.
